(19) United States
(12) Patent Application Publication
(10) Pub. No.: US 2025/0387228 A1
(43) Pub. Date: Dec. 25, 2025

(54) PROSTHETIC VALVE WITH POSTERIOR POSITIONING

(71) Applicant: INNOVALVE BIO MEDICAL LTD., Ramat-Gan (IL)

(72) Inventors: Guy Shimel, Tel Aviv-Jaffa (IL); Shai Karni, Rehovot (IL); Eyal Baror, Bet Nehemia (IL)

(21) Appl. No.: 19/323,183

(22) Filed: Sep. 9, 2025

Related U.S. Application Data
(63) Continuation of application No. PCT/IB2024/052304, filed on Mar. 10, 2024.
(60) Provisional application No. 63/451,261, filed on Mar. 10, 2023.

(51) Int. Cl. A61F 2/24

(52) U.S. Cl. CPC A61F 2/243; A61F 2/2418

(57) ABSTRACT

Apparatus and methods are described including a delivery device that delivers a valve frame to a native valve and positions the valve frame such that a center of the valve frame is off center with respect to a center of an annular plane of the valve annulus, and is positioned toward a posterior side of the annular plane. Arms of the valve frame are deployed among the chords of the native valve. The valve frame is rotated, such as to cause the arms to pull the leaflets of the native valve radially inwards. A frame body of the valve frame radially expands, such as to trap the native valve leaflets, and such that the valve frame is deployed off center with respect to the center of the annular plane. Other applications are also described.

### CROSS-REFERENCES TO RELATED APPLICATIONS

The present application is a Continuation of PCT Application No. PCT/IB2024/052304 to Shimel et al. (published as WO 24/189509), filed Mar. 10, 2024, entitled “Prosthetic mitral valve with posterior positioning,” which claims priority from U.S. Provisional Patent Application 63/451,261 to Shimel et al., filed Mar. 10, 2023, entitled “Positioning of prosthetic mitral valve,” which is incorporated herein by reference.

### FIELD OF EMBODIMENTS OF THE INVENTION

The present invention relates to medical apparatus and methods, and specifically to apparatus and methods for percutaneously delivering a medical device to a deployment location within a subject's body, such as an atrioventricular valve.

### BACKGROUND

The human heart is a muscular organ that pumps deoxygenated blood through the lungs to oxygenate the blood and pumps oxygenated blood to the rest of the body by contractions of four chambers.

After having circulated in the body, deoxygenated blood from the body enters the right atrium through the vena cava(s). In a healthy subject, the right atrium contracts, pumping the blood through the tricuspid valve into the right ventricle. The right ventricle contracts, pumping the blood through the pulmonary semi-lunar valve into the pulmonary artery which splits to two branches, one for each lung. The blood is oxygenated while passing through the lungs, and reenters the heart via the left atrium. The left atrium contracts, pumping the oxygenated blood through the mitral valve into the left ventricle. The left ventricle contracts, pumping the oxygenated blood through the aortic valve into the aorta to be distributed to the rest of the body. The tricuspid valve closes during right ventricle contraction, so that backflow of blood into the right atrium is prevented. Similarly, the mitral valve closes during left ventricle contraction, so that backflow of blood into the left atrium is prevented. The mitral valve and the tricuspid valve are known as atrioventricular valves, each of these valves controlling the flow of blood between an atrium and a ventricle.

In the mitral valve, the mitral annulus defines a mitral valve orifice. An anterior leaflet and a posterior leaflet extend from the mitral annulus. The leaflets are connected by chords to papillary muscles within the left ventricle. During ventricular diastole, in a healthy subject, the left atrium contracts to pump blood into the left ventricle through the mitral valve orifice. The blood flows through the orifice, pushing the leaflets apart and into the left ventricle with little resistance. In a healthy subject, the leaflets of the aortic valve are kept closed by blood pressure in the aorta.

During ventricular systole, the left ventricle contracts to pump blood into the aorta through the aortic valve, the leaflets of which are pushed open by the blood flow. In a healthy subject, the mitral annulus contracts, pushing the leaflets inwards and reducing the area of the mitral valve orifice by about 20% to 30%. The leaflets coapt to accommodate the excess leaflet surface area, producing a coaptation surface that constitutes a seal. The pressure of blood in the left ventricle pushes against the ventricular surfaces of the leaflets, tightly pressing the leaflets together at the coaptation surface so that a tight, leak-proof seal is formed.

An effective seal of the mitral valve during ventricular systole depends on a sufficient degree of coaptation. Improper coaptation may be caused by any number of physical anomalies that allow leaflet prolapse (for example, elongated or ruptured chords, or weak papillary muscles) or prevent coaptation (for example, short chords, or small leaflets). There are also pathologies that lead to a mitral valve insufficiency, including collagen vascular disease, ischemic mitral regurgitation (resulting, for example, from myocardial infarction, chronic heart failure, or failed/unsuccessful surgical or catheter revascularization), myxomatous degeneration of the leaflets, and rheumatic heart disease. Mitral valve regurgitation leads to many complications including arrhythmia, atrial fibrillation, cardiac palpitations, chest pain, congestive heart failure, fainting, fatigue, low cardiac output, orthopnea, paroxysmal nocturnal dyspnea, pulmonary edema, shortness of breath, and sudden death.

There are various medical devices that are configured to be delivered in a minimally-invasive procedure, in which a delivery device is used to deliver the device percutaneously (through a puncture in the skin) to a deployment location at which the device is to be deployed. Many such medical devices are deployed within the subject's vasculature and/or within the subject's heart. For example, such medical devices may include prosthetic valves (e.g., a prosthetic mitral valve, a prosthetic aortic valve, and/or a prosthetic tricuspid valve), valve repair devices (e.g., an annuloplasty ring or an edge-to-edge device, such as a mitral-leaflet clip), stents, hole-closure devices, and/or intravascular simulation devices. Typically, depending on the deployment location, larger medical devices are inserted into the subject's vasculature via the femoral vein or the femoral artery, while smaller devices may also be inserted via the radial vein or the radial artery, or another vein or artery. During delivery of the medical devices to the deployment location, the medical devices are typically maintained in a radially-constrained (i.e., crimped) configuration within the delivery device. The medical devices are radially expanded to their deployment configurations when disposed at the deployment location. In some cases, the medical devices are configured to self-expand, while in other cases the medical devices are radially expanded in an active manner, e.g., via balloon expansion.

There are various medical devices that are configured to be implanted at an atrioventricular valve (such as the mitral valve) and/or within the left ventricle. For example, a prosthetic mitral valve may be deployed to replace the native mitral valve. Or, a mitral valve repair device, such as an annuloplasty ring or a mitral-leaflet clip, may be deployed to repair an unhealthy mitral valve. Some such devices are implanted in an open surgery procedure. Others are implanted in a minimally-invasive procedure, in which a delivery device is used to deliver the device percutaneously to the mitral valve and/or the left ventricle. One approach for percutaneous delivery of a device to the mitral valve and/or the left ventricle is the transeptal approach. Using the transeptal approach, the delivery device is typically inserted into the femoral vein and then advanced through the subject's vena cava and from there through the right atrium and to the interatrial septum. The delivery device is then made to penetrate the interatrial septum, and is directed toward the mitral valve from within the left atrium.

Although there are many prosthetic mitral valves and mitral valve repair devices that are under development for the treatment of impaired mitral valves, to date, there is no effective transcatheter mitral valve replacement technology, and transcatheter mitral valve repair tends to produce imperfect outcomes. Surgery (whether mitral valve replacement or repair) carries substantial side effects, and is not suitable for all patients. In addition, with current treatment modalities, even if mitral regurgitation is corrected, left ventricular function (as measured using parameters such as ejection fraction) tends not to improve and even deteriorates.

### SUMMARY OF EMBODIMENTS

In accordance with some applications of the present invention, a delivery device is advanced from a subject's vena cava (e.g., via the inferior vena cava, or via the superior vena cava) into the subject's right atrium, and from there into the subject's left atrium, via the interatrial septum. The distal end of the delivery device is advanced toward the native mitral valve, and is typically advanced through leaflets of the native mitral valve and into the left ventricle. Typically, the delivery device is used to deliver a prosthetic mitral valve to be deployed at the subject's native mitral valve.

For some applications, the prosthetic mitral valve includes a valve frame having a valve frame body, which is deployed with the center of the valve frame off center with respect to the center of an annular plane of the valve annulus, and lies toward the posterior side of the annular plane. Typically, the prosthetic mitral valve is thereby configured to cause blood flow therethrough to be off center with respect to the center of the annular plane. Alternatively or additionally, the valve frame body is deployed angled toward the posterior side of the left ventricle, with a plane defined by a ventricular end of the valve frame at least partially facing a posterior wall of the left ventricle. Typically, the prosthetic mitral valve is thereby configured to cause blood flow therethrough to be directed toward the posterior wall of the left ventricle.

It is noted that prior art prosthetic mitral valves are typically implanted (either through open-heart surgery or via a transcatheteral approach) at the center of the annular plane, such that blood flow through the prosthetic mitral valve is not off center with respect to the center of the annular plane. Typically, by the valve frame of the present disclosure being deployed at the subject's mitral valve such that the center of the valve frame lies toward the posterior side of the annular plane, blood flow through the valve leaflets from the atrium to the left ventricle is off center with respect to the center of the annular plane. Blood flow from the atrium to the left ventricle being off center with respect to the center of the annular plane generates efficient blood flow from the left ventricle and towards the aorta (in a similar manner to blood flow through a healthy native mitral valve).

Typically, by the valve frame of the present disclosure being deployed at the subject's mitral valve such that the valve frame is angled toward the posterior side of the left ventricle, with the plane defined by the ventricular end of the valve frame at least partially facing the posterior wall of the left ventricle, much of the blood flow through the valve leaflets from the atrium to the left ventricle is directed toward the posterior wall of the left ventricle. Blood flow from the atrium to the left ventricle being directed toward the posterior wall generates efficient blood flow from the left ventricle and towards the aorta (in a similar manner to blood flow through a healthy native mitral valve).

For some applications, the subject's native anatomy is used to facilitate one or both of the above-described techniques. Typically, the valve frame becomes anchored to the subject's native mitral valve, inter alia, by rotating at least a portion of the valve frame, such as to cause the arms to pull the leaflets of the native valve radially inwards, by recruiting at least a portion of the chords of the native mitral valve, and subsequently, causing the frame body of the valve frame to radially expand, such as to trap the native valve leaflets. Typically, both the anterior and posterior native leaflets are trapped by the valve frame. Since the posterior leaflet is shorter than the anterior leaflet, in some cases, its anchoring serves as a pivot and causes the valve frame (a) to be deployed off center with respect to the center of the annular plane, with the valve frame positioned toward the posterior side of the annular plane, and/or (b) to be deployed angled toward the posterior side of the left ventricle with the plane defined by the ventricular end of the valve frame at least partially facing the posterior wall of the left ventricle.

There is therefore provided, in accordance with some embodiments of the present invention, apparatus for use with a prosthetic valve that is configured to be deployed within a native mitral valve of a mammalian subject, the native mitral valve including a valve annulus, valve leaflets, chords, and papillary muscles, the apparatus including:
a valve frame configured to support the prosthetic valve within the native mitral valve, the valve frame including a frame body and a plurality of arms that are configured to extend from the frame body; and
a delivery device configured to:
   deliver the valve frame to the native mitral valve;
   position the valve frame such that a center of the valve frame is off center with respect to a center of an annular plane of the valve annulus, and lies toward a posterior side of the annular plane;
   subsequently, deploy the arms among the chords of the native mitral valve;
   subsequently, rotate at least a portion of the valve frame, such as to cause the arms to pull the leaflets of the native valve radially inwards, by recruiting at least a portion of the chords of the native mitral valve; and
   subsequently, cause the frame body of the valve frame to radially expand, such as to trap the native valve leaflets, and such that the valve frame is deployed with the center of the valve frame off center with respect to the center of the annular plane, and is positioned toward the posterior side of the annular plane.

In some embodiments, the delivery device is configured to use a native posterior leaflet as a pivot to cause the valve frame to be deployed with the center of the valve frame off center with respect to the center of the annular plane, and positioned toward the posterior side of the annular plane.

In some embodiments, the delivery device is configured to cause blood flow through the prosthetic valve to be off center with respect to the center of the annular plane by causing the frame body of the valve frame to radially expand such that the valve frame is deployed with the center of the valve frame off center with respect to the center of the annular plane, and is positioned toward the posterior side of the annular plane.

In some embodiments, the delivery device is configured to position the valve frame such that the center of the valve frame is approximately aligned with a coaptation line of native anterior and posterior valve leaflets of the subject's mitral valve.

In some embodiments, by positioning the valve frame such that the center of the valve frame is approximately aligned with the coaptation line of the native anterior and posterior valve leaflets of the subject's mitral valve, the delivery device is configured to cause approximately equal numbers of anterior and posterior chords to be captured when the portion of the valve frame is rotated.

In some embodiments, the delivery device is further configured to:
position the valve frame such that the valve frame is angled toward a posterior side of the left ventricle with a plane defined by a ventricular end of the valve frame at least partially facing a posterior wall of the left ventricle; and
cause the frame body of the valve frame to radially expand, such that the valve frame is deployed angled toward the posterior side of the left ventricle with the plane defined by the ventricular end of the valve frame at least partially facing the posterior wall of the left ventricle.

In some embodiments, the delivery device is configured to cause blood flow through the prosthetic valve to be directed toward the posterior wall of the left ventricle by causing the frame body of the valve frame to radially expand such that the valve frame is deployed angled toward the posterior side of the left ventricle with the plane defined by the ventricular end of the valve frame at least partially facing the posterior wall of the left ventricle.

In some embodiments, the delivery device is configured to cause the frame body of the valve frame to radially expand, such that the plane defined by the ventricular end of the valve frame forms an angle with respect to the annular plane of more than 5 degrees.

In some embodiments, the delivery device is configured to cause the frame body of the valve frame to radially expand, such that the plane defined by the ventricular end of the valve frame forms an angle with respect to the annular plane of more than 15 degrees.

In some embodiments, the delivery device is configured to cause the frame body of the valve frame to radially expand, such that the plane defined by the ventricular end of the valve frame forms an angle with respect to the annular plane of between 5 and 40 degrees.

In some embodiments, the delivery device is configured to cause the frame body of the valve frame to radially expand, such that the plane defined by the ventricular end of the valve frame forms an angle with respect to the annular plane of between 15 and 25 degrees.

There is further provided, in accordance with some embodiments of the present invention, a method for use with a prosthetic valve that is configured to be deployed within a native mitral valve of a heart of a mammalian subject, the native mitral valve including a valve annulus, valve leaflets, chords, and papillary muscles, the method including:
placing a valve frame within the subject's heart, the valve frame including a valve frame body and a plurality of arms that are configured to extend from the valve frame body;
positioning the valve frame such that a center of the valve frame is off center with respect to a center of an annular plane of the valve annulus, and is positioned toward a posterior side of the annular plane;
subsequently, deploying the arms among the chords of the native mitral valve;
subsequently, rotating at least a portion of the valve frame, such as to cause the arms to pull the leaflets of the native valve radially inwards, by recruiting at least a portion of the chords of the native mitral valve; and
subsequently, causing the frame body of the valve frame to radially expand, such as to trap the native valve leaflets, and such that the valve frame is deployed with the center of the valve frame off center with respect to the center of the annular plane, and is positioned toward the posterior side of the annular plane, with the valve frame supporting the prosthetic valve within the native mitral valve.

In some embodiments, causing the frame body of the valve frame to radially expand such that the valve frame is deployed with the center of the valve frame off center with respect to the center of the annular plane, and is positioned toward the posterior side of the annular plane includes using a native posterior leaflet as a pivot to cause the valve frame to be deployed with the center of the valve frame off center with respect to the center of the annular plane, and positioned toward the posterior side of the annular plane.

In some embodiments, causing the frame body of the valve frame to radially expand such that the valve frame is deployed with the center of the valve frame off center with respect to the center of the annular plane, and is positioned toward the posterior side of the annular plane includes causing blood flow through the prosthetic valve to be off center with respect to the center of the annular plane.

In some embodiments, positioning the valve frame such that the center of the valve frame is off center with respect to the center of the annular plane of the valve annulus, and is positioned toward the posterior side of the annular plane includes positioning the valve frame such that the center of the valve frame is approximately aligned with a coaptation line of native anterior and posterior valve leaflets of the subject's mitral valve.

In some embodiments, positioning the valve frame such that the center of the valve frame is approximately aligned with the coaptation line of native anterior and posterior valve leaflets of the subject's mitral valve includes causing approximately equal numbers of anterior and posterior chords to be captured when the portion of the valve frame is rotated.

In some embodiments, the method further includes:
positioning the valve frame such that the valve frame is angled toward a posterior side of a left ventricle of the subject's heart with a plane defined by a ventricular end of the valve frame at least partially facing a posterior wall of the left ventricle; and
causing the frame body of the valve frame to radially expand, such that the valve frame is deployed angled toward the posterior side of the left ventricle with the plane defined by the ventricular end of the valve frame at least partially facing the posterior wall of the left ventricle.

In some embodiments, causing the frame body of the valve frame to radially expand such that the valve frame is deployed angled toward the posterior side of the left ventricle includes causing blood flow through the prosthetic valve to be directed toward the posterior wall of the left ventricle.

In some embodiments, causing the frame body of the valve frame to radially expand such that the valve frame is deployed angled toward the posterior side of the left ventricle includes causing the frame body of the valve frame to radially expand, such that the plane defined by the ventricular end of the valve frame forms an angle with respect to the annular plane of more than 5 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of more than 15 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of between 5 and 40 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of between 15 and 25 degrees.

There is further provided, in accordance with some embodiments of the present invention, apparatus for use with a prosthetic valve that is configured to be deployed within a native mitral valve of a mammalian subject, the native mitral valve including a valve annulus, valve leaflets, chords, and papillary muscles, the apparatus including:
a valve frame configured to support the prosthetic valve within the native mitral valve, the valve frame including a frame body and a plurality of arms that are configured to extend from the frame body; and
a delivery device configured to:
   deliver the valve frame to the native mitral valve;
   position the valve frame such that the valve frame is angled toward a posterior side of the left ventricle with a plane defined by a ventricular end of the valve frame at least partially facing a posterior wall of the left ventricle;
   subsequently, deploy the arms among the chords of the native mitral valve;
   subsequently, rotate at least a portion of the valve frame, such as to cause the arms to pull the leaflets of the native valve radially inwards, by recruiting at least a portion of the chords of the native mitral valve; and
   subsequently, cause the frame body of the valve frame to radially expand, such as to trap the native valve leaflets, and such that the valve frame is deployed angled toward the posterior side of the left ventricle with the plane defined by the ventricular end of the valve frame at least partially facing the posterior wall of the left ventricle.

In some embodiments, the delivery device is configured to use a native posterior leaflet as a pivot to cause the valve frame to be deployed angled toward the posterior side of the left ventricle with the plane defined by the ventricular end of the valve frame at least partially facing the posterior wall of the left ventricle.

In some embodiments, the delivery device is configured to cause blood flow through the prosthetic valve to be directed toward the posterior wall of the left ventricle by causing the frame body of the valve frame to radially expand such that the valve frame is deployed angled toward the posterior side of the left ventricle with the plane defined by the ventricular end of the valve frame at least partially facing the posterior wall of the left ventricle.

In some embodiments, the delivery device is configured to cause the frame body of the valve frame to radially expand, such that the plane defined by the ventricular end of the valve frame forms an angle with respect to the annular plane of more than 5 degrees.

In some embodiments, the delivery device is configured to cause the frame body of the valve frame to radially expand, such that the plane defined by the ventricular end of the valve frame forms an angle with respect to the annular plane of more than 15 degrees.

In some embodiments, the delivery device is configured to cause the frame body of the valve frame to radially expand, such that the plane defined by the ventricular end of the valve frame forms an angle with respect to the annular plane of between 5 and 40 degrees.

In some embodiments, the delivery device is configured to cause the frame body of the valve frame to radially expand, such that the plane defined by the ventricular end of the valve frame forms an angle with respect to the annular plane of between 15 and 25 degrees.

In some embodiments, the delivery device is further configured to:
position the valve frame such that a center of the valve frame is off center with respect to a center of an annular plane of the valve annulus, and is positioned toward a posterior side of the annular plane; and
cause the frame body of the valve frame to radially expand such that the valve frame is deployed with the center of the valve frame off center with respect to the center of the annular plane, and is positioned toward the posterior side of the annular plane.

In some embodiments, the delivery device is configured to cause blood flow through the prosthetic valve to be off center with respect to the center of the annular plane by causing the frame body of the valve frame to radially expand such that the valve frame is deployed with the center of the valve frame off center with respect to the center of the annular plane, and is positioned toward the posterior side of the annular plane.

In some embodiments, the delivery device is configured to position the valve frame such that the center of the valve frame is approximately aligned with a coaptation line of native anterior and posterior valve leaflets of the subject's mitral valve.

In some embodiments, by positioning the valve frame such that the center of the valve frame is approximately aligned with the coaptation line of the native anterior and posterior valve leaflets of the subject's mitral valve, the delivery device is configured to cause approximately equal numbers of anterior and posterior chords to be captured when the portion of the valve frame is rotated.

There is further provided, in accordance with some embodiments of the present invention, a method for use with a prosthetic valve that is configured to be deployed within a native mitral valve of a heart of a mammalian subject, the native mitral valve including a valve annulus, valve leaflets, chords, and papillary muscles, the method including:
placing a valve frame within the subject's heart, the valve frame including a valve frame body and a plurality of arms that are configured to extend from the valve frame body;
positioning the valve frame such that the valve frame is angled toward a posterior side of a left ventricle of the subject's heart with a plane defined by a ventricular end of the valve frame at least partially facing a posterior wall of the left ventricle;
subsequently, deploying the arms among the chords of the native mitral valve;
subsequently, rotating at least a portion of the valve frame, such as to cause the arms to pull the leaflets of the native valve radially inwards, by recruiting at least a portion of the chords of the native mitral valve; and
subsequently, causing the frame body of the valve frame to radially expand, such as to trap the native valve leaflets, and such that the valve frame is deployed angled toward the posterior side of the left ventricle with the plane defined by the ventricular end of the valve frame at least partially facing the posterior wall of the left ventricle, with the valve frame supporting the prosthetic valve within the native mitral valve.

In some embodiments, causing the frame body of the valve frame to radially expand such that the valve frame is deployed angled toward the posterior side of the left ventricle with the plane defined by the ventricular end of the valve frame at least partially facing the posterior wall of the left ventricle includes using a native posterior leaflet as a pivot to cause the valve frame to be deployed angled toward the posterior side of the left ventricle with the plane defined by the ventricular end of the valve frame at least partially facing the posterior wall of the left ventricle.

In some embodiments, causing the frame body of the valve frame to radially expand such that the valve frame is deployed angled toward the posterior side of the left ventricle includes causing blood flow through the prosthetic valve to be directed toward the posterior wall of the left ventricle.

In some embodiments, causing the frame body of the valve frame to radially expand such that the valve frame is deployed angled toward the posterior side of the left ventricle includes causing the frame body of the valve frame to radially expand such that the plane defined by the ventricular end of the valve frame forms an angle with respect to the annular plane of more than 5 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of more than 15 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of between 5 and 40 degrees.

In some embodiments, causing the frame body of the valve frame to radially expand, such that the valve frame is deployed angled toward the posterior side of the left ventricle includes causing the frame body of the valve frame to radially expand, such that the plane defined by the ventricular end of the valve frame forms an angle with respect to the annular plane of between 15 and 25 degrees.

In some embodiments, the method further includes:
positioning the valve frame such that a center of the valve frame is off center with respect to a center of an annular plane of the valve annulus, and is positioned toward a posterior side of the annular plane; and
causing the frame body of the valve frame to radially expand such that the valve frame is deployed with the center of the valve frame off center with respect to the center of the annular plane, and is positioned toward the posterior side of the annular plane.

In some embodiments, causing the frame body of the valve frame to radially expand such that the valve frame is deployed with the center of the valve frame off center with respect to the center of the annular plane, and is positioned toward the posterior side of the annular plane includes causing blood flow through the prosthetic valve to be off center with respect to the center of the annular plane.

In some embodiments, positioning the valve frame such that the center of the valve frame is off center with respect to the center of the annular plane of the valve annulus and is positioned toward the posterior side of the annular plane includes positioning the valve frame such that the center of the valve frame is approximately aligned with a coaptation line of native anterior and posterior valve leaflets of the subject's mitral valve.

In some embodiments, positioning the valve frame such that the center of the valve frame is approximately aligned with the coaptation line of native anterior and posterior valve leaflets of the subject's mitral valve includes causing approximately equal numbers of anterior and posterior chords to be captured when the portion of the valve frame is rotated.

The present invention will be more fully understood from the following detailed description of applications thereof, taken together with the drawings, in which:

### BRIEF DESCRIPTION OF THE DRAWINGS

FIGS. 1A and 1B are schematic illustrations showing a delivery device being advanced toward a subject's left ventricle, in accordance with some applications of the present invention;

FIGS. 2A, 2B, and 2C are schematic illustrations of inner and outer steerable catheters of a delivery device, in accordance with some applications of the present invention;

FIGS. 3A and 3B are schematic illustrations showing a capsule of a delivery device, in accordance with some applications of the present invention;

FIGS. 4A, 4B, and 4C are schematic illustrations of proximal and distal capsule portions of the delivery device, in accordance with some applications of the present invention;

FIG. 5 is a schematic illustration of a stage and handle portion of the delivery device, in accordance with some applications of the present invention;

FIGS. 6A and 6B are schematic illustrations of a delivery device, in accordance with some applications of the present invention;

FIGS. 7A, 7B, 7C, and 7D are schematic illustrations of a valve frame that is configured to support a prosthetic valve within a subject's native atrio-ventricular valve, the figures showing the valve frame disposed in a non-radially-constrained configuration, in accordance with some applications of the present invention;

FIGS. 8A, 8B, 8C, 8D, and 8E are schematic illustrations of respective steps of the deployment of a prosthetic mitral valve via a transseptal approach, in accordance with some applications of the present invention;

FIG. 9A is a schematic illustration showing an anterior-posterior cross section of a subject's left ventricle;

FIG. 9B is a schematic illustration of an anterior-posterior cross section of a subject's left ventricle with a prosthetic mitral valve frame, which supports valve leaflets, deployed at the mitral valve, such that the center of the valve frame is off center with respect to the center of the annular plane, and is positioned toward the posterior side of the annular plane, in accordance with some applications of the present invention; and

FIG. 9C is a schematic illustration of an anterior-posterior cross section of a subject's left ventricle, with a prosthetic mitral valve frame, which supports valve leaflets, deployed at the mitral valve, such that the valve frame is angled toward the posterior side of the left ventricle (i.e., such that the leaflets point toward the posterior wall of the left ventricle), in accordance with some applications of the present invention.

### DETAILED DESCRIPTION OF EMBODIMENTS

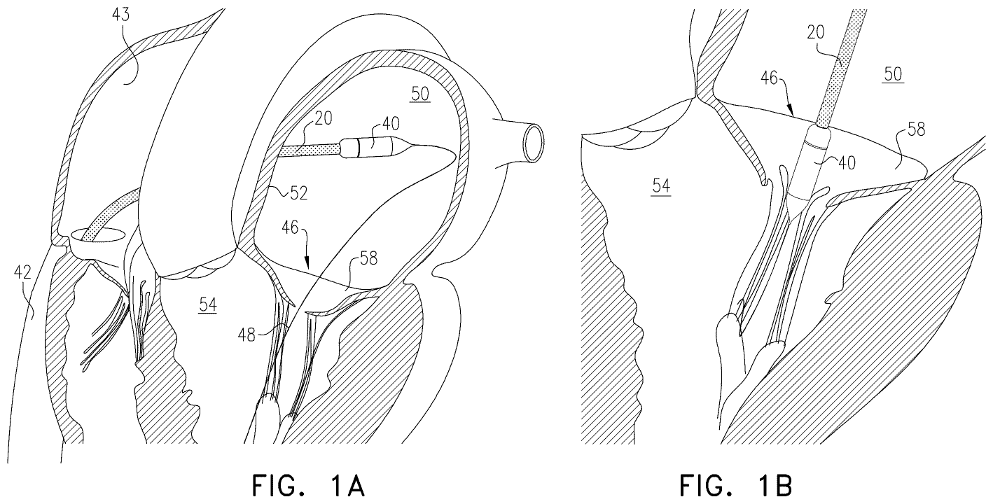

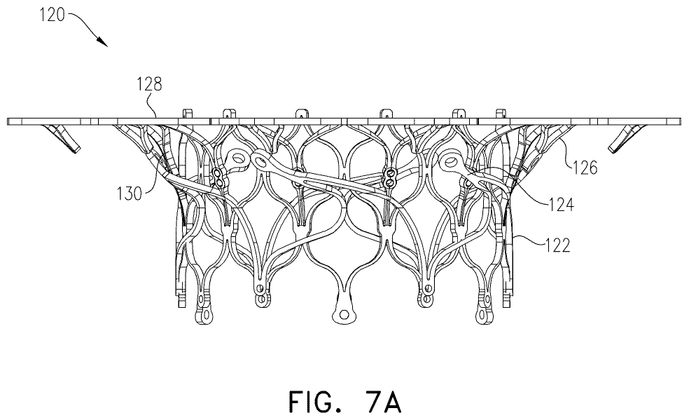

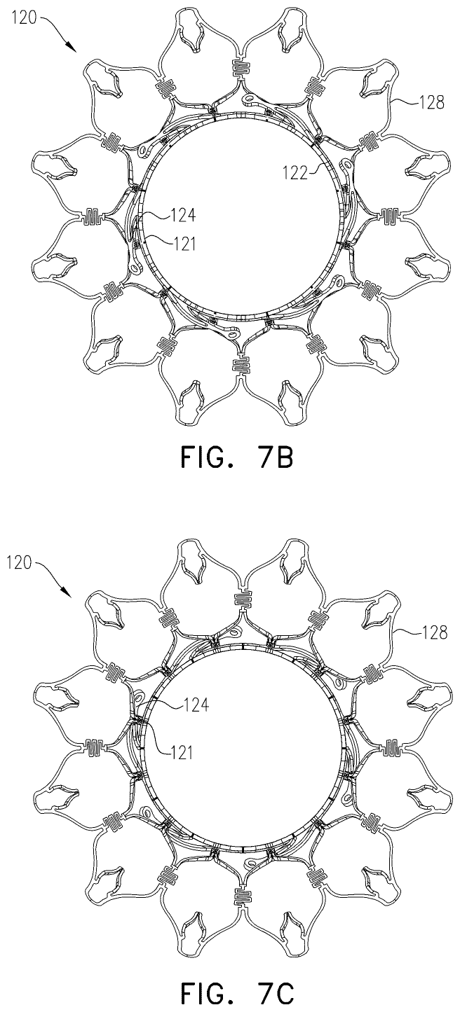

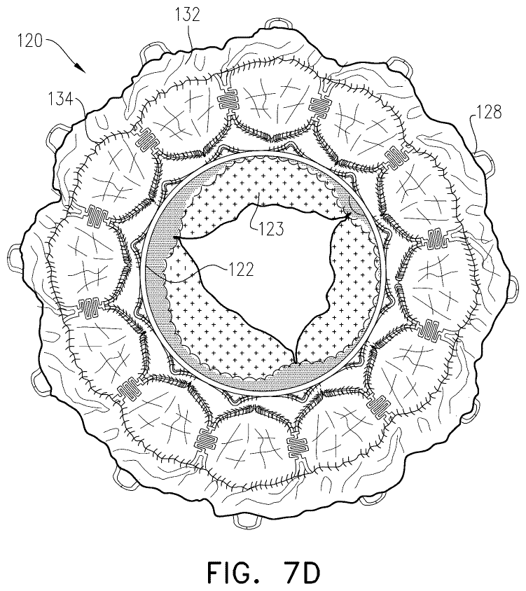

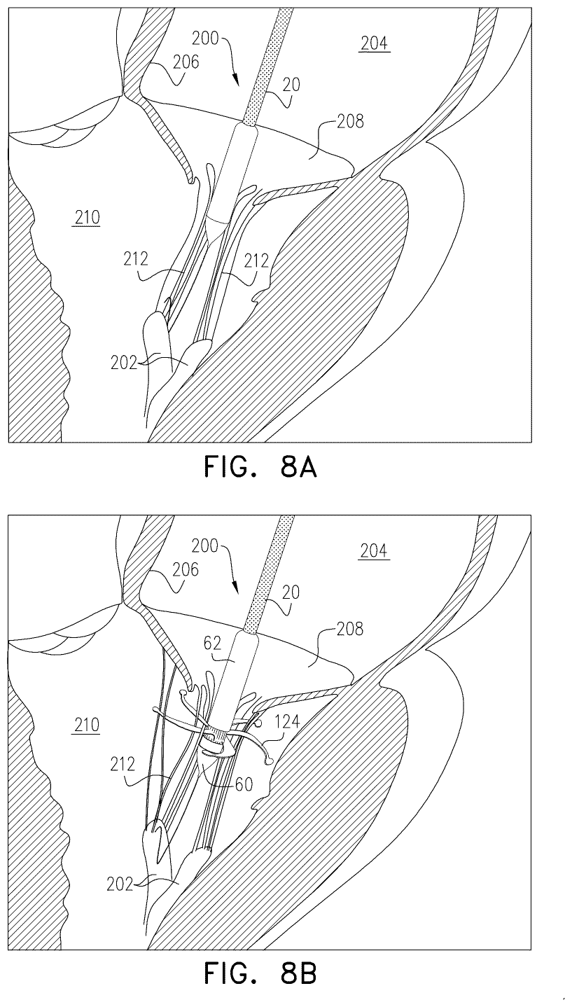

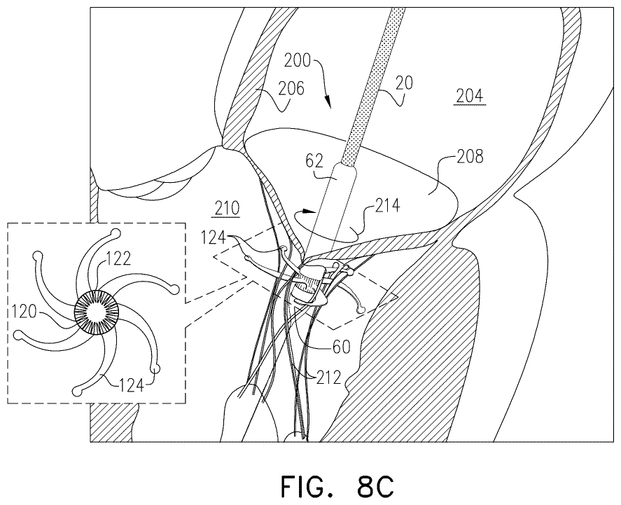

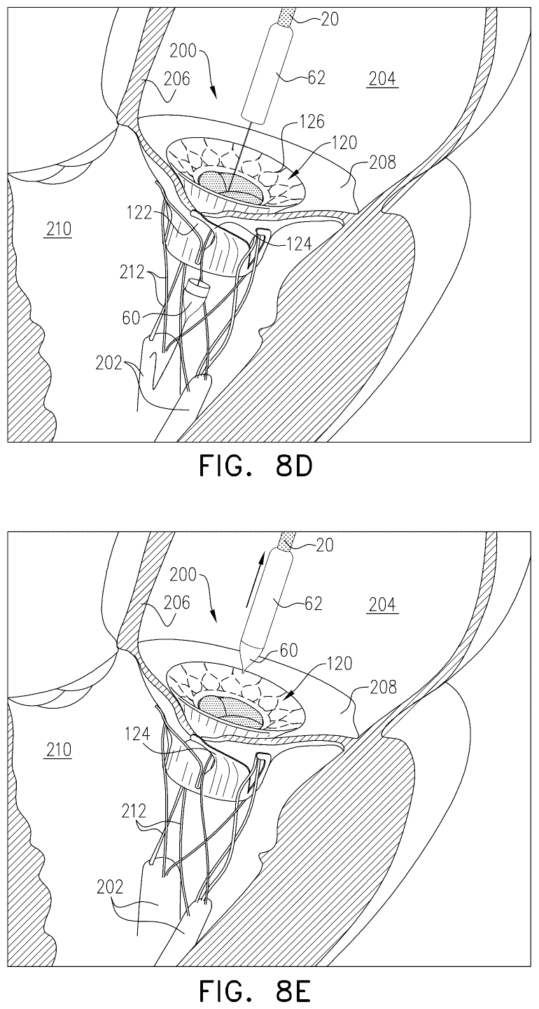

Reference is now made to FIGS. 1A and 1B, which are schematic illustrations showing the advancement of a delivery device 20 toward a subject's native mitral valve 46 and/or left ventricle 54, via a transseptal delivery approach, in accordance with some applications of the present invention. As shown in FIG. 1A, the distal end of delivery device 20 is typically advanced from the subject's vena cava 42 into the subject's right atrium 43, and from there into the subject's left atrium 50, via the interatrial septum 52. The distal end of the delivery device is advanced toward the native mitral valve, and is typically advanced through leaflets 58 of the native mitral valve and into left ventricle 54, as shown in FIG. 1B. For some applications, delivery device 20 is guided toward the subject's native mitral valve 46 over a guidewire 48. Typically, the delivery device is used to deliver a percutaneously-implantable medical device, such as a prosthetic mitral valve (as shown schematically in FIGS. 7A-8C), a mitral valve repair device (such as an annuloplasty ring or a mitral-leaflet clip), artificial chordae tendineae, and/or a different percutaneously-implantable medical device.

For some applications, the delivery device includes capsule 40 at its distal end. Typically, the percutaneously-implantable medical device is held in a crimped (i.e., radially-constrained) configuration inside the capsule, during delivery of the medical device to the subject's mitral valve and/or left ventricle. Further typically, in order to deploy the device at the subject's mitral valve and/or left ventricle, the medical device is released from the capsule, as described in further detail hereinbelow. For some applications, the medical device is a self-expandable medical device that is configured to self-expand radially upon being released from the capsule. For example, the medical device may include a shape-memory alloy (such as nitinol) that is shape set to a desired radially-expanded configuration. Alternatively or additionally, the device may actively be radially expanded after being released from the capsule (e.g., via balloon expansion). For some applications, a distal portion of the medical device is first released from the capsule, and a proximal portion of the medical device is subsequently released from the capsule, as described in further detail hereinbelow.

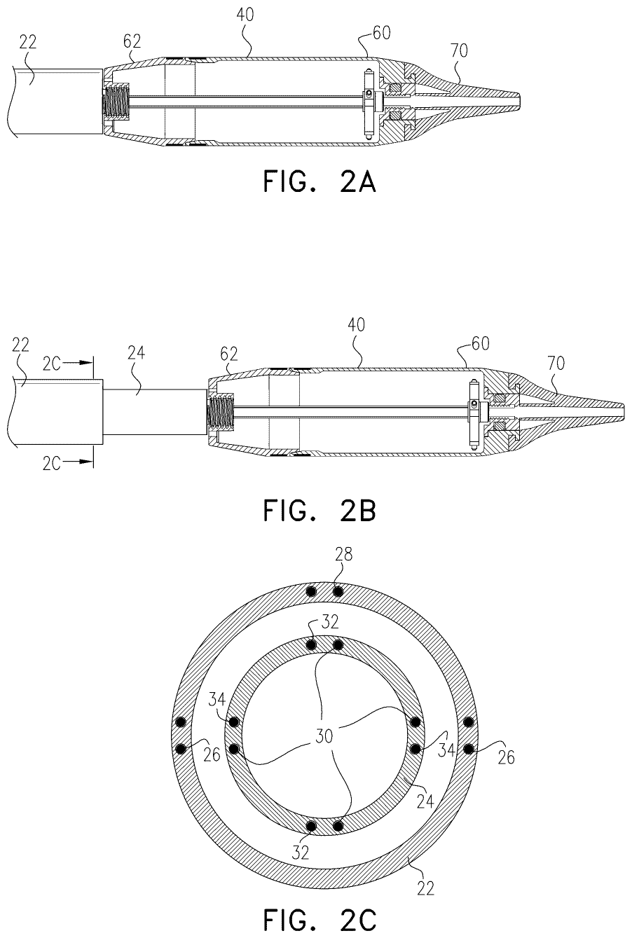

Reference is now made to FIGS. 2A, 2B, and 2C, which are schematic illustrations of an outer steerable catheter 22 and an inner steerable catheter 24 of delivery device 20, in accordance with some applications of the present invention. FIGS. 2A and 2B show side views and FIG. 2C shows a cross-sectional view of the outer and inner steerable catheters. As shown in the transition from FIG. 2A to FIG. 2B, typically the inner steerable catheter is axially-slidable with respect to the outer steerable catheter. Typically, during advancement of delivery device 20 from the subject's vena cava 42 into the subject's left atrium 50, via the interatrial septum 52 (anatomy shown in FIGS. 1A-1B), the distal end of the inner steerable catheter is disposed inside the outer steerable catheter, as shown in FIG. 2A. Further typically, once the distal end of the outer steerable catheter is disposed inside the left atrium, the inner steerable catheter is advanced out of the distal end of the outer steerable catheter (i.e., the configuration shown in FIG. 2B) and then steered toward the subject's mitral valve and/or left ventricle. For some applications, the inner steerable catheter is configured to be steered independently of the outer steerable catheter once the inner steerable catheter has been advanced out of the distal end of the outer steerable catheter.

For some applications, the outer steerable catheter includes first and second steering deflection cables 26 that are configured to be operated by a user to steer the distal end of the outer catheter through a first outer-steerable-catheter deflection plane, toward the subject's interatrial septum. Alternatively (embodiment not shown), the outer catheter includes only a single steering-deflection cable 26 that is configured to be operated by a user to steer the distal end of the outer steerable catheter through the first outer-steerable-catheter deflection plane toward the subject's interatrial septum. Typically, in addition to one or more steering deflection cables 26, the outer catheter includes a height-adjustment deflection cable 28. Typically, the height-adjustment deflection cable 28 is configured to be operated by a user to deflect the distal end of the outer steerable catheter from within the left atrium toward the roof of the left atrium, by steering the tip of the outer steerable catheter through a second outer-steerable-catheter deflection plane. Typically, the second outer-steerable-catheter deflection plane is perpendicular to the first outer-steerable-catheter deflection plane. Accordingly, height-adjustment deflection cable 28 is typically disposed at a 90 degree angle with respect to steering-deflection cable(s) 26, as shown in FIG. 2C.

For some applications, steering deflection cables 26 are configured to steer the distal end of the outer steerable catheter through the first outer-steerable-catheter deflection plane through an angle of between 0 degrees and more than 60 degrees, or more than 75 degrees (e.g., 0-90 degrees). For some applications, height-adjustment deflection cable 28 is configured to steer the distal end of the outer steerable catheter through the second outer-steerable-catheter deflection plane through an angle of between 0 degrees and more than 30 degrees, or more than 40 degrees (e.g., 0-45 degrees), to deflect the distal end of the outer steerable catheter from within the left atrium toward the roof of the left atrium.

It is noted that in FIG. 2C, each steering deflection cable is shown as being doubled. This is because, typically, each steering deflection cable follows a first path from a proximal end of the catheter to the distal end of the catheter, before following a return path from the distal end of the catheter to the proximal end of the catheter.

It is noted that within the left atrium, the inner steerable catheter typically needs to maneuver through a curve of approximately 90 degrees. This is because the inner steerable catheter is advanced out of the outer steerable catheter after the outer steerable catheter has penetrated the interatrial septum. Thus, the tip of the inner steerable catheter typically advances from the outer steerable catheter facing a lateral direction and must be steered to face an inferior-anterior direction in order to advance toward the mitral valve. Typically, the outer steerable catheter is made to penetrate the interatrial septum below the roof of the atrium, as shown in FIGS. 1A-1B (e.g., at a posterior-inferior, or a posterior-superior location), since the septum is thinner and more easily penetrated at this location. As described above, the height-adjustment deflection cable 28 is configured to be operated by a user to deflect the distal end of the outer steerable catheter from within the left atrium toward the roof of the left atrium. Typically, this provides the inner steerable catheter with a greater height within which to maneuver through the above-described curve, such that the curve is less acute, and also provide height for the capsule to deploy above the annulus.

Typically, inner steerable catheter 22 includes one or more steering deflection cables 30. For some applications, the inner steerable catheter includes (a) a first set 32 of one or more (e.g., a pair of) steering deflection cables that are configured to be operated by a user to steer the distal end of the inner steerable catheter through a first inner-steerable-catheter deflection plane, toward the subject's mitral valve, and (b) a second set 34 of one or more (e.g., a pair of) steering deflection cables that are configured to be operated by a user to steer the distal end of the inner steerable catheter through a second inner-steerable-catheter deflection plane, such as to align the distal end of the inner steerable catheter with the subject's mitral valve.

For some applications, first set 32 of steering deflection cables is configured to steer the distal end of the inner steerable catheter through the first inner-steerable-catheter deflection plane through an angle of between 0 degrees and more than 80 degrees, or more than 100 degrees (e.g., 120 degrees). For some applications, second set 34 of steering deflection cables is configured to steer the distal end of the inner steerable catheter through the second inner-steerable-catheter deflection plane through an angle of at least between-45 degrees and +45 degrees, such as to align the distal end of the inner steerable catheter with the subject's mitral valve. Typically, set 32 of the steering deflection cable(s) is disposed at a 90 degree angle with respect to second set 34 of steering deflection cable(s), as shown in FIG. 2C.

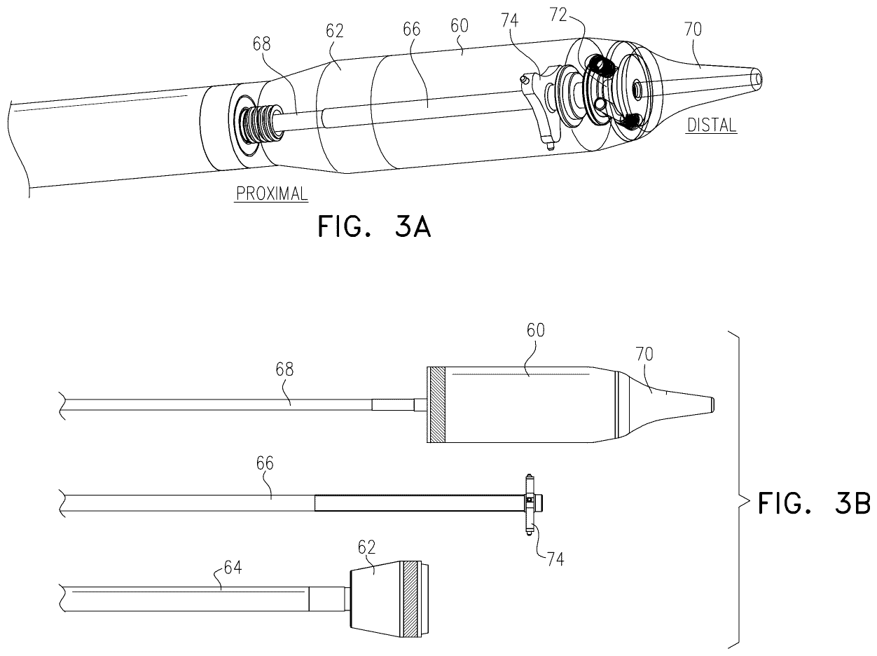

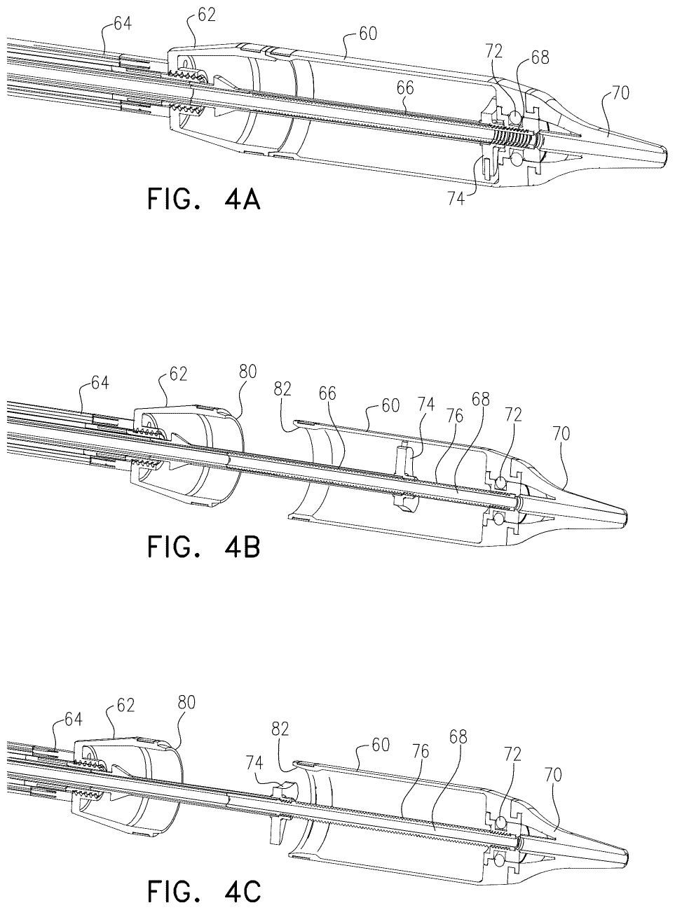

Reference is now made to FIGS. 3A and 3B, which are schematic illustrations showing capsule 40 of delivery device 20, in accordance with some applications of the present invention. Typically, the medical device is held in a crimped (i.e., radially-constrained) configuration inside the capsule, during delivery of the medical device to a deployment location (such as the subject's mitral valve and/or left ventricle). Further typically, in order to deploy the device at the deployment location, the medical device is released from the capsule. It is noted that a capsule as shown in FIGS. 3A-3B (as well as in FIGS. 4A-4C) may be used with any medical device that is delivered to deployment location within a subject's body in a crimped configuration and is not limited to being used with devices that are deployed within the mitral valve and/or left ventricle. For example, a capsule as shown in FIGS. 3A-3B (as well as in FIGS. 4A-4C) could be used with a medical device that is delivered to a subject's aorta, vena cava tricuspid valve, right ventricle, right atrium, right ventricle, pulmonary vein, pulmonary artery, etc.

For some applications, the capsule includes a distal capsule portion 60 configured to maintain a distal portion of the medical device in the radially-constrained configuration during delivery of the medical device to the deployment location, and a proximal capsule portion 62 configured to maintain a proximal portion of the medical device in a radially-constrained configuration during delivery of the medical device to the deployment location. Typically, the proximal and distal portions are reversibly couplable to each other, as described in further detail hereinbelow. For some applications, the capsule additionally includes a tapered distal tip 70 that is configured to facilitate advancement of the capsule into the subject's vasculature, and, subsequently, acts as a dilator to advance through the interatrial septum. Typically, the distal tip is made of a soft material, such that the tip is atraumatic and does not cause injury to tissue of the subject during advancement of the delivery device to the deployment location. The distal tip typically allows the advancement of the system on a guidewire and its soft material complies with the guidewire direction.

For some applications, an outer shaft 64, a medial shaft 66, and an inner shaft 68 are all disposed within inner steerable catheter 24 (shown in FIGS. 2B-2C). The outer shaft is typically coupled to proximal capsule portion 62, such that axial motion of the outer shaft relative to the medial shaft and the inner shaft transmits axial motion to the proximal capsule portion relative to the medial shaft and the inner shaft. In order to release the proximal portion of the medical device from within the proximal capsule portion, the outer shaft is typically retracted axially, proximally relative to the medial shaft and the inner shaft, which causes the proximal capsule portion to be retracted from over the proximal portion of the medical device. (It is noted that, rather than retracting the outer shaft, the relative proximal motion of the outer shaft with respect to the medial shaft and the inner shaft may be effected by advancing the medial shaft and the inner shaft distally relative to the outer shaft.)

Inner shaft 68 is typically coupled to the distal capsule portion 60 such that that axial motion of the inner shaft transmits axial motion to the distal capsule portion. (It is noted that rotational motion of the distal capsule portion is typically separated from rotational motion of the inner shaft via a bearing mechanism 72, as described in further detail hereinbelow with reference to FIGS. 4A-4C.) For some applications, the delivery device includes a distal device interface 74, which is configured to secure a distal portion of the medical device at a fixed axial location with respect to the medial shaft, so long as the distal portion of the medical device is held within the distal capsule portion. For some applications, the distal device interface is a flange which extends radially from the medial shaft, as shown. In order to release the distal portion of the medical device from within the distal capsule portion, the inner shaft is typically advanced axially and distally (typically using the techniques described hereinbelow with reference to FIGS. 4A-4C) relative to the medial shaft. This causes the distal capsule portion to be advanced distally relative to the distal device interface. Once the proximal end of the distal capsule portion is advanced beyond the distal device interface, the distal portion of the medical device is typically released from the distal device interface (typically, via radial self-expansion of the distal portion of the medical device, and/or by another mechanism as described hereinabove).

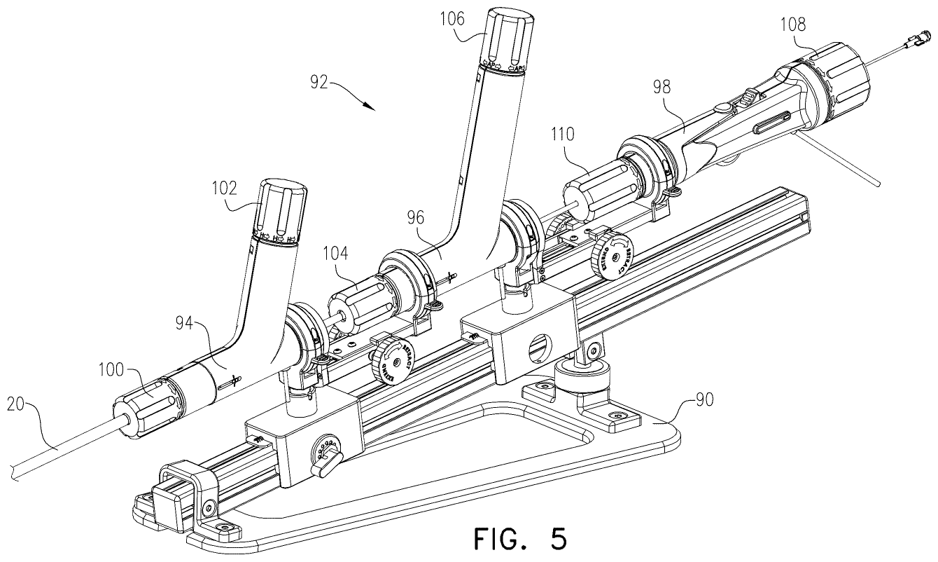

Reference is now made to FIGS. 4A, 4B, and 4C, which are schematic illustrations of proximal capsule portion 62 and distal capsule portion 60 of the delivery device at respective stages of the advancement of distal capsule portion 60 with respect to proximal capsule portion 62, in accordance with some applications of the present invention. In some cases, it is desirable to advance the distal capsule portion 60 with respect to proximal capsule portion 62 in a precisely controlled manner. For example, when used with a prosthetic mitral valve frame as shown in FIGS. 7A-7D, it may be desirable to initially release an intermediate portion of the valve frame (e.g., radially-expandable arms of the valve frame) from being covered by the distal capsule portion, without fully releasing the entire distal portion of the valve frame. Typically, in order to allow a physician to maintain precise control of the advancement of distal capsule portion 60 with respect to proximal capsule portion 62, the physician uses a rotational control mechanism (e.g., mechanism 108 shown in FIG. 5) and the rotational motion of the rotational control mechanism is converted to axial motion of inner shaft 68 (which is coupled to the distal capsule portion). For some such applications, the conversion of the rotational motion to the axial motion of inner shaft 68 is effected at the distal end of the inner shaft, and typically within the capsule. It is noted that if the conversion of the rotational motion to the axial motion of inner shaft 68 were to be effected at the proximal end of the inner shaft, the axial motion of the inner shaft would then need to be transmitted along the entire length of the inner shaft before being transmitted to the distal capsule portion, which can result in imprecise transmission of axial motion to the distal capsule portion. By contrast, by converting the rotational motion to the axial motion of inner shaft 68 at the distal end of the inner shaft (in accordance with some applications of the present invention), the axial motion does not need to be transmitted along the entire length of the inner shaft before being transmitted to the distal capsule portion. Rather, the axial motion is transmitted from within the capsule to the distal capsule portion.

For some applications, inner shaft 68 defines a threaded outer surface 76 at its distal end, and the inner surface of distal device interface 74 (which is described hereinabove is typically a flange) and/or medial shaft 66 is correspondingly threaded. The threaded inner surface of distal device interface 74 and/or medial shaft 66 acts as a nut, such that rotation of the distal end of the inner shaft causes the inner shaft to advance distally with respect to the distal device interface 74. As described hereinabove, typically the distal device interface 74 secures the distal end of the medical device and further typically, axial motion of the inner shaft is transmitted to the distal capsule portion. Therefore, the advancement of the inner shaft with respect to the distal device interface 74 causes the distal capsule portion to advance relative to a distal end of the medical device. As described hereinabove, for some applications, the distal capsule portion includes a bearing mechanism 72. The bearing mechanism is configured to separate rotational motion of the distal capsule portion from rotational motion of the inner shaft. Thus, rotation of the inner shaft causes the distal capsule portion to be advanced distally relative to the distal end of the medical device, but without causing the distal capsule portion to rotate.

Typically, once the medical device has been released from within capsule 40, the proximal and distal portions of the capsule are re-coupled to each other before being retracted from within the subject's body. For some applications, the capsule includes a guide portion defined by at least one of the distal and proximal capsule portions. The guide portion is configured to guide the distal and proximal capsule portions back into their coupled configuration, subsequent to the medical device having been deployed. For example, as shown in FIG. 4B-4C, for some applications the proximal capsule portion defines a lip 80 at its distal end, and the distal capsule portion defines a corresponding lip 82 at is proximal end, with lips 80 and 82 being shaped such as to slide into place with respect to each other. Alternatively, only one of the capsule portions defines a lip, and the lip is configured to receive the other capsule portion (embodiment not shown). Typically, when the proximal and distal portions are correctly coupled to each other they are shaped such as to define a substantially smooth outer surface. In this manner, during advancement of the capsule to the medical device deployment location, the capsule is atraumatic and does not cause damage to tissue of the subject. Similarly, during retraction of the capsule from the medical device deployment location, the capsule is atraumatic and does not cause damage to tissue of the subject or to the deployed medical device. For some applications, the above-mentioned lip is formed as a complete ring (as shown). For some applications (not shown), a lip that is generally as described above is split into multiple, separate, arc-shaped, segments. For example, the lip may be formed from 4 arc-shaped segments, spaced 90 degrees apart from each other and each covering an arc of 30 degrees. In this way the medical device may be released before the entire capsule is removed, hence saving on the height that is required to release the medical device.

FIG. 5 is a schematic illustration of a stage 90 and handle portion 92 of the delivery device, in accordance with some applications of the present invention. For some applications, the handle portion include a first handle 94 configured to control steering of outer steerable catheter 22, a second handle 96 configured to control steering of inner steerable catheter 24, and a deployment handle 98 configured to control the release of the medical device from capsule 40.

Typically, first handle 94 includes a first rotational control mechanism 100 for controlling steering deflection cables 26 (which are configured to be operated by a user to steer the distal end of the outer steerable catheter through a first outer-steerable-catheter deflection plane toward the subject's interatrial septum). Further typically, first handle 94 includes a second rotational control mechanism 102 for controlling height-adjustment deflection cable 28 (which is configured to be operated by a user to deflect the distal end of the outer steerable catheter from within the left atrium toward the roof of the left atrium, by steering the tip of the outer steerable catheter through a second outer-steerable-catheter deflection plane).

Typically, second handle 96 includes a first rotational control mechanism 104 for controlling first set 32 of steering deflection cables (which are configured to be operated by a user to steer the distal end of the inner steerable catheter through a first inner-steerable-catheter deflection plane, toward the subject's mitral valve). Further typically, second handle 96 includes a second rotational control mechanism 106 for controlling second set 34 of steering deflection cables (which are configured to be operated by a user to steer the distal end of the inner steerable catheter through a second inner-steerable-catheter deflection plane, such as to align the distal end of the inner steerable catheter with the subject's mitral valve).

As described hereinabove, the deployment handle typically includes a rotational control mechanism 108 for controlling axial motion of the distal capsule portion 60. Further typically, the deployment handle includes a second rotational control mechanism 110 for controlling axial motion of proximal capsule portion 62. Typically, the handle portion includes a plurality of flushing ports, via which respective catheter and shafts are flushed.

Typically, stage 90 is configured to position handle portion 92 and allows adjustments of position of the handle portion. For some applications, the stage is configured to facilitate quick attachment of the handle portion to the stage without requiring any screws, e.g., via a snap-lock mechanism. For some applications, the stage is configured to facilitate modification of the orientation of the handle portion during the procedure to allow realignment of the handle portion with respect to the percutaneous access point.

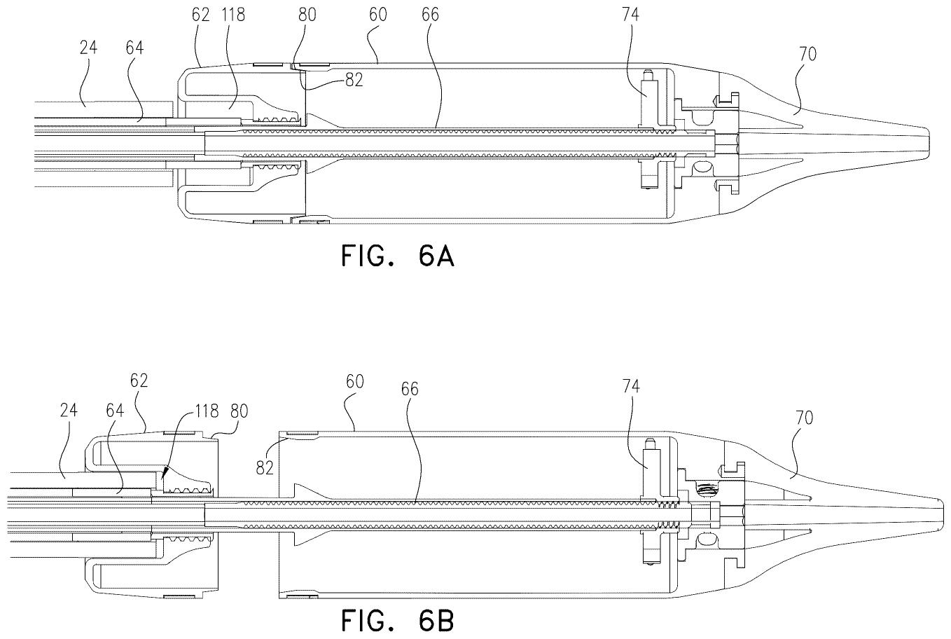

Reference is now made to FIGS. 6A and 6B, which are schematic illustrations of delivery device 20, in accordance with some applications of the present invention. In general, delivery device 20 as shown in FIGS. 6A and 6B is similar to that shown in FIGS. 1A-5, except for the differences described hereinbelow. For some applications, the proximal end of proximal capsule portion 62 defines a recess 118. Typically, the recess is sized such that, as the proximal capsule portion is retracted, proximal capsule portion is able to overlap with a distal end of a delivery catheter (e.g., inner steerable catheter 24 of delivery device 20, described hereinabove with reference to FIGS. 2A-2C.) Typically, if not for the recess, it would be necessary for there to be a gap between the distal end of the delivery catheter and the proximal capsule portion, in order to enable the proximal capsule portion to be retracted relative to the delivery catheter (e.g., in order to release the proximal end of the implantable device). By contrast, when the proximal capsule portion includes recess 118, the proximal capsule portion is typically disposed adjacent to distal end of the delivery catheter even before the proximal capsule portion is retracted (as shown in FIG. 6A). Alternatively, the proximal capsule portion partially overlaps with the distal end of the delivery catheter even before the proximal capsule portion is retracted (embodiment not shown). Subsequently, when the proximal capsule portion 62 is retracted, the proximal end of the proximal capsule portion is made to overlap (or to further overlap) with distal end of the delivery catheter, by the recess sliding over the distal end of the delivery catheter. Typically, recess 118 allows the device to occupy less space (e.g., less height) within the left atrium than would otherwise be required, by removing the need for there to be a gap between the distal end of the delivery catheter and the proximal capsule portion.

Reference is now made to FIGS. 7A, 7B, 7C, which are schematic illustrations of respective views of a valve frame 120, the figures showing the valve frame in its non-radially-constrained configuration, in accordance with some applications of the present invention. FIG. 7A shows a side view of the valve frame, FIG. 7B shows a bottom view (i.e., a view from a ventricular end of the valve frame), and FIG. 7C shows a top view (i.e., a view from an atrial end of the valve frame). Reference is also made to FIG. 7D, which is a schematic illustration of valve frame 120, with valve leaflets 123 coupled to the valve frame, in accordance with some applications of the present invention.

Typically, the valve frame includes a valve-frame body 121. For some applications, valve-frame body 121 includes a cylindrical part 122, as well as an atrial part 126. Typically, the cylindrical part is configured to support the prosthetic valve within the native atrio-ventricular valve. For example, leaflets 123 of the prosthetic valve may be sutured to the cylindrical part, and/or may be otherwise coupled to the cylindrical part, e.g., as shown in FIG. 7D. Typically, atrial part 126 is configured to be deployed at least partially within the subject's atrium. For some applications, atrial part 126 includes a disc-shaped portion 128 (also referred to herein as a flange) and a frustoconical portion 130.

Typically, the disc-shaped portion of the atrial part is configured to seal the valve frame with respect to tissue on the atrial side of the mitral annulus, and is further configured to prevent migration of the valve frame into the left ventricle. The frustoconical portion typically extends from the disc-shaped portion of the atrial part to the outer surface of the cylindrical part. For some applications, the inclusion of the frustoconical portion between the disc-shaped portion and the cylindrical part (as opposed to directly coupling the disc-shaped portion to the cylindrical part) reduces a likelihood of regurgitation around the outside of the cylindrical part.

For some applications, the cylindrical part and the atrial part are formed as separate pieces from one another and are coupled to each other, for example, via stitching, gluing, welding, and/or another method. Alternatively, the cylindrical part and the atrial part are portions of a single integrally-formed piece.

Typically, valve frame 120 is made of a shape-memory material (e.g., a shape-memory alloy, such as nitinol and/or copper-aluminum-nickel), which is covered on one or both sides with a covering material 132 (shown in FIG. 7D), e.g., a fabric and/or a polymer (such as expanded polytetrafluoroethylene (ePTFE), or woven, knitted, mesh and/or braided polyester). Typically, the shape-memory material of cylindrical part 122 and atrial part 126 is shaped into a stent-like structure that comprises struts and/or cells of the shape-memory material. The covering material is typically coupled to the shape-memory material via stitches 134 (shown in FIG. 7D). It is noted that FIGS. 7A-7C show valve frame 120 in the absence of valve leaflets 123 and covering material 132 for illustrative purposes. However, valve leaflets 123, and covering material 132 may be observed in FIG. 7D.

For some applications, a plurality of chord-recruiting arms 124 (e.g., more than two and/or fewer than twelve arms) extend from a portion of valve-frame body 121 that is configured to be placed within the subject's ventricle. For example, four chord-recruiting arms or six chord-recruiting arms may extend from the valve-frame body. For some applications, a single chord-recruiting arm 124 extends from a portion of valve-frame body 121 that is configured to be placed within the subject's ventricle. Typically, the chord-recruiting arms extend from cylindrical part 122 of valve-frame body 121. Further typically, the chord-recruiting arms extend from a ventricular end of the cylindrical part (i.e., the end of the valve frame body that is configured to be placed within the ventricle). Typically, in a non-radially constrained configuration of the valve frame (which the valve frame typically assumes when neither the valve frame body nor the chord-recruiting arms are constrained by the delivery device), the arms extend radially from the valve-frame body, in addition to extending axially from the ventricular end of the valve-frame body toward an atrial end of the valve-frame body (i.e., the end of the valve frame body that is configured to be placed within the atrium). Further typically, the arms curve around outside of the valve-frame body in a given circumferential direction of curvature.

It is noted that descriptions herein of the arms extending from the valve-frame body in a given direction should not be interpreted as excluding additional directions in which the arms are oriented. Rather, the arms being described (or claimed) as extending radially from the valve-frame body should be interpreted as meaning that the orientation of the arms with respect to the valve-frame body includes a radial component. It is typically the case that, in addition to extending radially from the valve-frame body, the arms curve circumferentially, and in some cases the orientation of the arms includes an axial component. For some applications, at least along a portion of the arms, and at least in certain configurations of the arms, the arms are disposed tangentially with respect to the valve-frame body.

Typically, valve frame 120 with prosthetic valve leaflets 123 disposed therein is delivered to the native atrio-ventricular valve, via delivery device 20 (which is typically as described hereinabove), and the delivery device is configured to maintain the valve frame and the prosthetic valve in radially-constrained configurations (i.e., “crimped” configurations) during the delivery. In accordance with respective applications, the valve frame is delivered transapically (i.e., via the apex of the left ventricle), transseptally (i.e., via the vena cava, the right atrium, and the interatrial septum, as described in detail with reference to FIGS. 8A-8C), and/or via a different delivery path. For some applications, when a distal end of the delivery device is disposed within the subject's ventricle, chord-recruiting arms 124 are deployed among chords of the native atrio-ventricular valve. Typically, the chord-recruiting arms are deployed among chords of the native atrio-ventricular valve by releasing the chord-recruiting arms from the delivery device, the chord-recruiting arms being shape set to extend from the valve-frame body, upon being released from the delivery device. For some applications, additional techniques are used in order to cause the chord-recruiting arms to become deployed among chords of the native atrio-ventricular valve by releasing the chord-recruiting arms from the delivery device. For example, the valve frame may include lever elements, which are configured to cause the chord-recruiting arms to extend radially. Alternatively or additionally, the arms are coupled to the cylindrical part of the valve frame via stitches, the stitches acting as hinges, such that the arms pivot about the stitches with respect to the cylindrical part, as described hereinbelow. Typically, the chord-recruiting arms are released from the delivery device while the valve-frame body is still maintained in an at least partially radially-constrained configuration by the delivery device. Typically, the valve frame is rotated while the chord-recruiting arms and the valve-frame body are configured in the aforementioned configuration. Therefore, in the present application, the configuration of the chord-recruiting arms when the valve-frame body is still maintained in an at least partially radially-constrained configuration by the delivery device but the chord-recruiting arms have been released from the delivery device is referred to as the “rotation configuration” of the chord-recruiting arms.

Reference is now made to FIGS. 8A, 8B, 8C, 8D and 8E, which are schematic illustrations of respective steps of the delivery and deployment of a prosthetic mitral valve, via a transseptal approach, in accordance with some applications of the present invention. Typically, the prosthetic mitral valve includes a valve frame body as described hereinabove, with prosthetic valve leaflets 123 sutured to the cylindrical part, and/or otherwise coupled to cylindrical part 122 of the valve frame, e.g., as shown in FIG. 7D. As described hereinabove, in accordance with respective applications, the prosthetic mitral valve is delivered transseptally (i.e., via the vena cava, the right atrium, and the interatrial septum), transapically (i.e., via the apex of the left ventricle), and/or via a different delivery path. FIGS. 8A-8E shows steps of delivery and deployment of a prosthetic mitral valve, via the transseptal approach, by way of illustration and not limitation.

Typically, delivery device 20 is guided toward the subject's native mitral valve 200 over a guidewire 202. The distal end of delivery device 20 is typically advanced into the subject's left atrium 204, via the interatrial septum 206. The distal end of the delivery device is advanced toward the native mitral valve, and is advanced through leaflets 208 of the native mitral valve and into left ventricle 210, as shown in FIG. 8A. When the distal end of the delivery device is disposed within the left ventricle, chord-recruiting arms 124 are allowed to at least partially radially expand, and assume their rotation configurations, as shown in FIG. 8B. For some applications, the arms are allowed to assume non-radially-constrained configurations by releasing the arms from being radially constrained by the delivery device, e.g., by partially retracting proximal capsule portion 62, and/or by partially advancing distal capsule portion 60. Typically, the chord-recruiting arms are shape set to extend radially from valve-frame body 121 and to curve circumferentially around the valve-frame body (e.g., in the clockwise direction, as shown), upon assuming their rotation configurations. For some applications, the chord-recruiting arms are further configured to extend axially toward the subject's atrium. Typically, the chord-recruiting arms are configured to become deployed among chords 212 of the native mitral valve upon being released from the delivery device.

As shown in FIG. 8C, subsequent to the chord-recruiting arms 124 being deployed among chords of the native mitral valve, at least a portion of the valve frame is rotated in the direction of arrow 214, such as to cause chord-recruiting arms 124 to (a) pull the native atrioventricular valve radially inward toward the valve frame, and (b) twist the native atrioventricular valve around the valve frame, by recruiting and deflecting at least a portion of the chords. Typically, the chord-recruiting arms 124 are configured to curve in a given circumferential direction with respect to the longitudinal axis of the valve frame. For example, the arms may curve in a clockwise direction or in a counter-clockwise direction with respect to the longitudinal axis of the valve frame. Typically, subsequent to chord-recruiting arms 124 being deployed among chords of the native mitral valve, the valve frame is rotated in the same circumferential direction as the direction of the circumferential curvature of the arms. In the example shown in FIG. 8C, the arms curve in the clockwise circumferential direction (as viewed from left atrium 204), and the valve frame is rotated in this direction.

For some applications, prior to rotating the valve frame in the same circumferential direction as the direction of the circumferential curvature of the arms, the valve frame is rotated in the opposite circumferential direction. For some applications, the delivery device 20 is configured such as to automatically perform the initial rotation of the valve frame through a given angle against the direction of circumferential curvature of the arm, and to subsequently rotate the valve frame though a predetermined angle in the direction of the circumferential curvature of the arms. For some applications, in the rotation configuration of the arms (shown in FIGS. 8B-8C), the outer surfaces of each of the arms has a smooth, convex curvature that extends along substantially the full length of the arm, such that during the initial rotation (against the direction of circumferential curvature of the arm) the chords slide over the outer surfaces of the arm without be recruited or caught by the arm. For some applications, by virtue of the arms being shaped in this manner, the initial rotation of the valve frame causes a relatively large number of chords to be positioned such as to be recruited by each of the arms in the subsequent rotation step. During the subsequent rotation of the valve frame (in the direction of the circumferential curvature of the arms, e.g., the direction of arrow 214 as shown in FIG. 8C), the chords are recruited and deflected by the arms. Typically, in the rotation configuration of the arms (shown in FIGS. 8B-8C), the inner surface of the arm has a concave curvature and the chords are recruited within the space defined by the concave curvature, during the subsequent rotation by the valve frame.

Subsequent to chord-recruiting arms 124 having been released and valve frame 120 having been rotated, valve-frame body 121 (i.e., cylindrical part 122 and atrial part 126 of the valve frame) is allowed to assume its non-radially-constrained configurations. For some applications, the atrial part is allowed to assume its non-radially-constrained configuration by releasing the atrial part from the delivery device, e.g., by retracting proximal capsule portion 62. For some applications, the cylindrical part is allowed to assume its non-radially-constrained configuration by releasing the cylindrical part from the delivery device, e.g., by advancing distal capsule portion 60. FIG. 8D shows both cylindrical part 122 and atrial part 126 in their non-radially-constrained (i.e., radially-expanded) configurations. Typically, by the valve-frame body assuming its non-radially-constrained configuration, the valve-frame body is configured to trap the native valve leaflets 208 in a partially closed and twisted configuration, to thereby at least partially seal a space between the native mitral valve and the prosthetic valve. For example, the cylindrical part may be configured to radially expand such as to trap the native valve leaflets between the cylindrical part and the chord-recruiting arms, and/or the atrial part may be configured to radially expand such as to trap the native valve leaflets between the atrial portion and the chord-recruiting arms. For some applications, the trapping of native valve leaflets 208 in a partially closed and twisted configuration is achieved by trapping the chords (which are attached to the leaflets) in twisted configurations. Subsequent to the above described steps being performed, delivery device 20 is typically then retracted in its entirety from the subject's left atrium, as indicated in FIG. 8E.

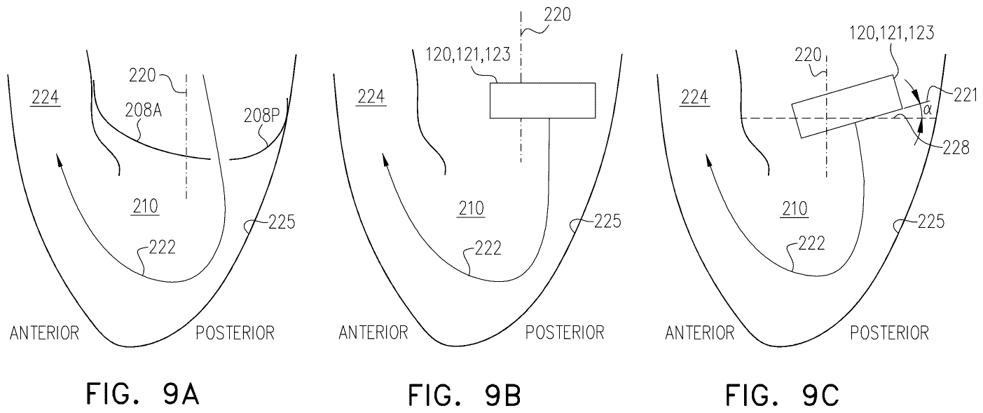

Reference is now made to FIG. 9A, which is a schematic illustration of an anterior-posterior cross section of a healthy left ventricle 210, in the absence of any implanted device. As is well known, the anterior leaflet 208A is substantially larger than the posterior leaflet 208P. As a result, the coaptation line of the leaflets (i.e., the line at which the anterior and posterior leaflets coapt with each other) is off center with respect to the center 220 of the annular plane, and is positioned on the posterior side of the left ventricle. Typically, the fact that the coaptation line of the leaflets is off center with respect to the center 220 of the annular plane causes blood flow from atrium to the left ventricle to be off center with respect to the center of the annular plane. As indicated by blood flow arrow 222 in FIG. 9A, blood flow from atrium to the left ventricle being off center with respect to the center of the annular plane generates efficient blood flow from the left ventricle and towards the aorta 224. Moreover, the anterior leaflet directs much of the blood flow from the atrium to the left ventricle toward the posterior wall 225, which further generates efficient blood flow from the left ventricle and towards the aorta 224.

Reference is now made to FIG. 9B, which is a schematic illustration of an anterior-posterior cross section of a subject's left ventricle with prosthetic mitral valve frame 120 (which supports valve leaflets 123) deployed at the mitral valve, such that the center of the valve frame is off center with respect to the center 220 of the annular plane, and is positioned toward the posterior side of the annular plane, in accordance with some applications of the present invention. It is noted that prior art prosthetic mitral valves are typically implanted at the center of the annular plane, such that blood flow through the prosthetic mitral valve is not off center with respect to the center of the annular plane. Typically, by the valve frame of the present disclosure being deployed at the subject's mitral valve such that the center of the valve frame is positioned toward the posterior side of the annular plane, blood flow through the valve leaflets from the atrium to the left ventricle is off center with respect to the center of the annular plane. As indicated by blood flow arrow 222 in FIG. 9B, blood flow from the atrium to the left ventricle being off center with respect to the center of the annular plane generates efficient blood flow from the left ventricle and towards the aorta 224 (in a similar manner to blood flow through a healthy native mitral valve).

Reference is now made to FIG. 9C, which is a schematic illustration of an anterior-posterior cross section of a subject's left ventricle, with prosthetic mitral valve frame 120 (which supports valve leaflets 123) deployed at the mitral valve, such that the valve frame is angled toward the posterior side of the left ventricle (i.e., such that the leaflets open toward the posterior wall of the left ventricle), in accordance with some applications of the present invention. It is noted that prior art prosthetic mitral valves are typically implanted such that the plane defined by the valve frame is substantially parallel to the annular plane 228, such that blood flow through the prosthetic mitral valve is not directed toward the posterior wall. Typically, by the valve frame of the present disclosure being deployed at the subject's mitral valve such that the valve frame is angled toward the posterior side of the left ventricle, much of the blood flow through the valve leaflets from the atrium to the left ventricle is directed toward posterior wall 225. As indicated by blood flow arrow 222 in FIG. 9C, blood flow from the atrium to the left ventricle being directed toward the posterior wall generates efficient blood flow from the left ventricle and towards the aorta 224 (in a similar manner to blood flow through a healthy native mitral valve). For some applications, the valve frame is positioned and deployed angled toward the posterior side of the left ventricle, such that a plane 221 defined by the ventricular end of the valve frame at least partially faces a posterior wall of the left ventricle. For some applications, the valve frame is angled toward the posterior side of the left ventricle, such that plane 221 defined by the ventricular end of the valve frame forms an angle alpha with respect to annular plane 228 of more than 5 degrees (e.g., more than 15 degrees) and/or less than 40 degrees (e.g., less than 25 degrees), e.g., 5-40 degrees or 15-25 degrees.

It is noted that for some applications, the techniques described with reference to FIGS. 9B and 9C are combined. That is to say that, for some applications, prosthetic mitral valve frame 120 is deployed at a subject's mitral valve, such that (a) the center of the valve frame is off center with respect to the center 220 of the annular plane, and is positioned toward the posterior side of the annular plane, and (b) the valve frame is angled toward the posterior side of the left ventricle.

For some applications, the subject's native anatomy is used to facilitate one or both of the above-described techniques. As described above, typically, the valve frame becomes anchored to the subject's native mitral valve, inter alia, by rotating at least a portion of the valve frame, such as to cause the arms to pull the leaflets of the native valve radially inwards, by recruiting at least a portion of the chords of the native mitral valve, and subsequently, causing the frame body of the valve frame to radially expand, such as to trap the native valve leaflets. Typically, both the anterior and posterior native leaflets are trapped by the valve frame. Since the posterior leaflet is shorter than the anterior leaflet, in some cases, its anchoring serves as a pivot and causes the valve frame (a) to be deployed off center with respect to the center of the annular plane, with the valve frame positioned toward the posterior side of the annular plane, and/or (b) to be deployed angled toward the posterior side of the left ventricle with the plane defined by the ventricular end of the valve frame at least partially facing the posterior wall of the left ventricle.

For some applications, delivery device 20 is configured to position mitral valve frame with respect to the mitral valve annulus, such that the valve frame becomes deployed in a position and/or orientation as described in FIG. 9B and/or FIG. 9C. For some applications, prior to the valve frame being rotated (i.e., the step shown in FIG. 8C), the delivery device is configured to position the valve frame such that a center of the valve frame is approximately aligned with the coaptation line of the native anterior and posterior valve leaflets. Thus, when the valve frame is rotated approximately equal numbers of anterior and posterior chords are recruited by the chord-recruiting arms, such that when the valve frame is fully deployed, the valve frame is centered by the anterior and posterior chords that the center of the valve frame is off center with respect to the center 220 of the annular plane, and is positioned toward the posterior side of the annular plane.

As noted above with reference to delivery device 20, typically the inner steerable catheter 24 (shown in FIG. 2C) includes first set 32 and second set 34 of steering deflection cables. Typically, the first set 32 of steering deflection cables is configured to steer the distal end of the inner steerable catheter through a first inner-steerable-catheter deflection plane through an angle of between 0 degrees and more than 80 degrees, or more than 100 degrees (e.g., 120 degrees). For some applications, the second set 34 of steering deflection cables is configured to steer the distal end of the inner steerable catheter through a second inner-steerable-catheter deflection plane through an angle of at least between-45 degrees and +45 degrees. For some applications, the second set of steering deflection cables is configured to position the valve frame such that the valve frame is angled toward the posterior side of the left ventricle (as described with reference to FIG. 9C), prior to the deployment of the valve frame.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a prosthetic valve that is configured to be deployed within a native valve of a heart of a subject, the native valve including a valve annulus, valve leaflets, chords, and papillary muscles, the apparatus comprising:
a valve frame configured to support the prosthetic valve within the native valve, the valve frame comprising a frame body and a plurality of arms that are configured to extend from the frame body; and
a delivery device configured to:
deliver the valve frame to the native valve;
position the valve frame such that a center of the valve frame is off center with respect to a center of an annular plane of the valve annulus, and is positioned toward a posterior side of the annular plane;
subsequently, deploy the arms among the chords of the native valve;
subsequently, rotate at least a portion of the valve frame, such as to cause the arms to pull the leaflets of the native valve radially inwards, by recruiting at least a portion of the chords of the native valve; and
subsequently, cause the frame body of the valve frame to radially expand, such as to trap the native valve leaflets, and such that the valve frame is deployed with the center of the valve frame off center with respect to the center of the annular plane, and is positioned toward the posterior side of the annular plane.

2. The apparatus according to claim 1, wherein the delivery device is configured to use a native posterior leaflet as a pivot to cause the valve frame to be deployed with the center of the valve frame off center with respect to the center of the annular plane, and positioned toward the posterior side of the annular plane.

3. The apparatus according to claim 1, wherein the delivery device is configured to cause blood flow through the prosthetic valve to be off center with respect to the center of the annular plane by causing the frame body of the valve frame to radially expand such that the valve frame is deployed with the center of the valve frame off center with respect to the center of the annular plane, and is positioned toward the posterior side of the annular plane.

4. The apparatus according to claim 1, wherein the apparatus is for use with a native mitral valve, and the valve frame is configured to support the prosthetic valve within the native valve.

5. The apparatus according to claim 4, wherein the delivery device is configured to position the valve frame such that the center of the valve frame is approximately aligned with a coaptation line of native anterior and posterior valve leaflets of the native mitral valve.

6. The apparatus according to claim 5, wherein by positioning the valve frame such that the center of the valve frame is approximately aligned with the coaptation line of the native anterior and posterior valve leaflets of the native mitral valve, the delivery device is configured to cause approximately equal numbers of anterior and posterior chords to be captured when the portion of the valve frame is rotated.

7. The apparatus according to claim 1, wherein the delivery device is further configured to:
position the valve frame such that the valve frame is angled toward a posterior side of a ventricle of the subject's heart, with a plane defined by a ventricular end of the valve frame at least partially facing a posterior wall of the ventricle; and
cause the frame body of the valve frame to radially expand, such that the valve frame is deployed angled toward the posterior side of the ventricle with the plane defined by the ventricular end of the valve frame at least partially facing the posterior wall of the ventricle.

8. The apparatus according to claim 7, wherein the delivery device is configured to cause the frame body of the valve frame to radially expand, such that the plane defined by the ventricular end of the valve frame forms an angle with respect to the annular plane of more than 5 degrees.

9. A method for use with a prosthetic valve that is configured to be deployed within a native valve of a heart of a subject, the native valve including a valve annulus, valve leaflets, chords, and papillary muscles, the method comprising:
placing a valve frame within the subject's heart, the valve frame including a valve frame body and a plurality of arms that are configured to extend from the valve frame body;
positioning the valve frame such that a center of the valve frame is off center with respect to a center of an annular plane of the valve annulus, and is positioned toward a posterior side of the annular plane;
subsequently, deploying the arms among the chords of the native valve;
subsequently, rotating at least a portion of the valve frame, such as to cause the arms to pull the leaflets of the native valve radially inwards, by recruiting at least a portion of the chords of the native valve; and
subsequently, causing the frame body of the valve frame to radially expand, such as to trap the native valve leaflets, and such that the valve frame is deployed with the center of the valve frame off center with respect to the center of the annular plane, and is positioned toward the posterior side of the annular plane, with the valve frame supporting the prosthetic valve within the native valve.

10. The method according to claim 9, wherein causing the frame body of the valve frame to radially expand such that the valve frame is deployed with the center of the valve frame off center with respect to the center of the annular plane, and is positioned toward the posterior side of the annular plane comprises using a native posterior leaflet as a pivot to cause the valve frame to be deployed with the center of the valve frame off center with respect to the center of the annular plane, and positioned toward the posterior side of the annular plane.

11. The method according to claim 9, wherein causing the frame body of the valve frame to radially expand such that the valve frame is deployed with the center of the valve frame off center with respect to the center of the annular plane, and is positioned toward the posterior side of the annular plane comprises causing blood flow through the prosthetic valve to be off center with respect to the center of the annular plane.

12. The method according to claim 9, wherein the method is for use with a prosthetic valve that is configured to be deployed within a native mitral valve of the subject, and the valve frame is configured to support the prosthetic valve within the native mitral valve.

13. The method according to claim 12, wherein positioning the valve frame such that the center of the valve frame is off center with respect to the center of the annular plane of the valve annulus, and is positioned toward the posterior side of the annular plane comprises positioning the valve frame such that the center of the valve frame is approximately aligned with a coaptation line of native anterior and posterior valve leaflets of the native mitral valve.

14. The method according to claim 13, wherein positioning the valve frame such that the center of the valve frame is approximately aligned with the coaptation line of native anterior and posterior valve leaflets of the native mitral valve comprises causing approximately equal numbers of anterior and posterior chords to be captured when the portion of the valve frame is rotated.

15. The method according to claim 9, further comprising:
positioning the valve frame such that the valve frame is angled toward a posterior side of a ventricle of the subject's heart with a plane defined by a ventricular end of the valve frame at least partially facing a posterior wall of the ventricle; and
causing the frame body of the valve frame to radially expand, such that the valve frame is deployed angled toward the posterior side of the ventricle with the plane defined by the ventricular end of the valve frame at least partially facing the posterior wall of the ventricle.

16. The method according to claim 15, wherein causing the frame body of the valve frame to radially expand such that the valve frame is deployed angled toward the posterior side of the ventricle comprises causing blood flow through the prosthetic valve to be directed toward the posterior wall of the ventricle.

17. The method according to claim 15, wherein causing the frame body of the valve frame to radially expand such that the valve frame is deployed angled toward the posterior side of the ventricle comprises causing the frame body of the valve frame to radially expand, such that the plane defined by the ventricular end of the valve frame forms an angle with respect to the annular plane of more than 5 degrees.

18. A method for use with a prosthetic valve that is configured to be deployed within a native valve of a heart of a subject, the native valve including a valve annulus, valve leaflets, chords, and papillary muscles, the method comprising:
placing a valve frame within the subject's heart, the valve frame including a valve frame body and a plurality of arms that are configured to extend from the valve frame body;
positioning the valve frame such that the valve frame is angled toward a posterior side of a ventricle of the subject's heart with a plane defined by a ventricular end of the valve frame at least partially facing a posterior wall of the ventricle;
subsequently, deploying the arms among the chords of the native valve;
subsequently, rotating at least a portion of the valve frame, such as to cause the arms to pull the leaflets of the native valve radially inwards, by recruiting at least a portion of the chords of the native valve; and
subsequently, causing the frame body of the valve frame to radially expand, such as to trap the native valve leaflets, and such that the valve frame is deployed angled toward the posterior side of the ventricle with the plane defined by the ventricular end of the valve frame at least partially facing the posterior wall of the ventricle, with the valve frame supporting the prosthetic valve within the native valve.

19. The method according to claim 18, wherein causing the frame body of the valve frame to radially expand such that the valve frame is deployed angled toward the posterior side of the ventricle with the plane defined by the ventricular end of the valve frame at least partially facing the posterior wall of the ventricle comprises using a native posterior leaflet as a pivot to cause the valve frame to be deployed angled toward the posterior side of the ventricle with the plane defined by the ventricular end of the valve frame at least partially facing the posterior wall of the ventricle.

20. The method according to claim 18, wherein causing the frame body of the valve frame to radially expand such that the valve frame is deployed angled toward the posterior side of the ventricle comprises causing blood flow through the prosthetic valve to be directed toward the posterior wall of the ventricle.

21. The method according to claim 18, wherein causing the frame body of the valve frame to radially expand such that the valve frame is deployed angled toward the posterior side of the ventricle comprises causing the frame body of the valve frame to radially expand such that the plane defined by the ventricular end of the valve frame forms an angle with respect to the annular plane of more than 5 degrees.

22. The method according to claim 18, further comprising:
positioning the valve frame such that a center of the valve frame is off center with respect to a center of an annular plane of the valve annulus, and is positioned toward a posterior side of the annular plane; and
causing the frame body of the valve frame to radially expand such that the valve frame is deployed with the center of the valve frame off center with respect to the center of the annular plane, and is positioned toward the posterior side of the annular plane.
